(19) United States
(12) Patent Application Publication
(10) Pub. No.: US 2020/0294065 A1
(43) Pub. Date: Sep. 17, 2020

(54) System and Method for Identifying Suspicious Healthcare Behavior

(71) Applicant: Georgia Tech Research Corporation, atlanta, GA (US)

(72) Inventor: Musheer Ahmed, Atlanta, GA (US)

(21) Appl. No.: 16/082,110

(22) PCT Filed: Mar. 6, 2017
(86) PCT No.: PCT/US17/20916

§ 371 Date: Sep. 4, 2018

Related U.S. Application Data
(60) Provisional application No. 62/303,488, filed on Mar. 4, 2016.

(51) Int. Cl. G06Q 30/00; G06Q 10/10; G16H 50/20; G16H 10/60; G16H 50/70; G06Q 50/26; G06N 7/00

(52) U.S. Cl. CPC G06Q 30/0185; G06Q 10/10; G16H 50/20; G06N 7/005; G16H 50/70; G06Q 50/265; G16H 10/60

(57) ABSTRACT

Aspects of the disclosed technology include a method including receiving, by a processor, first healthcare data including a first plurality of features of a plurality of candidate profiles; identifying, by the processor, a profile calculation window; creating, by the processor, a reference profile of the first healthcare data over the profile calculation window by dimensionally reducing the first plurality of features, the reference profile including a normal subspace and a residual subspace; analyzing, by the processor, the residual subspace of the reference profile; and detecting, based on the analyzed residual subspace, suspicious behavior of one or more first candidate profiles of the plurality of candidate profiles based on a deviation of the one or more first candidate profiles within the residual subspace from an expected profile.

### CROSS-REFERENCE TO RELATED APPLICATIONS

This application claims the benefit of U.S. Provisional Application No. 62/303,488, filed Mar. 4, 2016, which is hereby incorporated herein by reference in its entirety.

### TECHNICAL FIELD

The present disclosure is related to detection of suspicious healthcare behavior, and more particularly, to providing systems and methods for identifying suspicious behavior within healthcare claims.

### BACKGROUND

Healthcare related fraud is estimated to cost up to several billions of dollars every year. Related art techniques to identify suspicious behavior in healthcare are supervised or semi-supervised and often require a labeled dataset. For example, some related art techniques are based on neural networks, decision trees, association rules, Bayesian networks, genetic algorithms, support vector machines, and structure pattern mining. However, many related art techniques fail to provide unsupervised detection of suspicious behavior.

For example, certain related art techniques attempt to prevent healthcare fraud by verifying the identity of a beneficiary. This may be accomplished through, for example, biometric verification, identification codes, multi-factor verification, or smart cards. However, such techniques are only able to prevent or limit medical identity theft and may require significant adoption costs or require widespread adoption rates in order to be effective.

Other related art techniques enforce predefined rules to detect suspicious behavior. For example, rules may be based on a distance between a beneficiary and a provider, patient readmission, healthcare service frequency, total amount of provider billings, or tracking medical codes applied to patient services. If a rule is triggered, the transaction or provider is flagged as suspicious. However, such pre-defined rules fail to detect new healthcare fraud schemes, or adapt to changes in healthcare practices.

A third set of related art techniques compare medical claims or treatments between peers to attempt to detect suspicious behavior. However, these related art techniques may require specific information on diagnoses, treatments, or sequence of procedures, or require customized equations which may not be adaptable to emerging schemes or changes in medical practice.

Accordingly, what is needed is a low supervision or unsupervised technique to identify suspicious behavior within medical care date.

### SUMMARY

Briefly described, and according to one embodiment, aspects of the present disclosure generally relate to a method of identifying suspicious activity. According to some embodiments, there is provided a method including: receiving, by a processor, first healthcare data including a first plurality of features of a plurality of candidate profiles; identifying, by the processor, a profile calculation window; creating, by the processor, a reference profile of the first healthcare data over the profile calculation window by dimensionally reducing the first plurality of features, the reference profile including a normal subspace and a residual subspace; analyzing, by the processor, the residual subspace of the reference profile; and detecting, based on the analyzed residual subspace, suspicious behavior of one or more first candidate profiles of the plurality of candidate profiles based on a deviation of the one or more first candidate profiles within the residual subspace from an expected profile.

The first healthcare data may include at least one of healthcare provider data, healthcare beneficiary data, and healthcare claim data.

The detecting the suspicious behavior may include determining, by the processor and based on the analyzed residual subspace, a deviation level of the one or more first candidate profiles, and the method may further include, in response to a deviation level of a candidate profile of the one or more first candidate profiles exceeding a threshold, automatically flagging, by the processor, the suspicious behavior of the candidate profile of the one or more first candidate profiles.

The detecting the suspicious behavior may include determining, by the processor and based on the analyzed residual subspace, a deviation level of the one or more first candidate profiles, and the method may further include, in response to a deviation level of a candidate profile of the one or more first candidate profiles exceeding a threshold, automatically stopping, by the processor, a payment for a claim related to the candidate profile of the one or more first candidate profiles.

The detecting the suspicious behavior may include determining, by the processor and based on the analyzed residual subspace, respective deviation levels of the one or more first candidate profiles, and the method may further include: determining, by the processor, respective costs associated with the one or more first candidate profiles; combining, by the processor, the respective deviation levels and the respective costs to determine respective expected values of the detected suspicious behavior; and ranking the one or more first candidate profiles based on the respective expected values.

The method may further include calculating, by the processor, deviation values of the detected one or more first candidate profiles; and ranking the detected one or more first candidate profiles based on the calculated deviation values.

The method may further include determining a basis for the ranking by calculating, by the processor, a normalized joint probability between a combination of categorical values within the first healthcare data to identify uncommon combinations.

The method may further include: determining a basis for the ranking by: identifying, by the processor, unusual numerical values within the first healthcare data; identifying, by the processor, uncommon categorical values within the first healthcare data; and calculating, by the processor, a normalized joint probability between a combination of categorical values within the first healthcare data to identify uncommon combinations, and providing the ranking and the identified numerical values, the uncommon categorical values, and the identified uncommon combinations for additional healthcare analysis.

The dimensionally reducing may include performing, by the processor, Principal Component Analysis (PCA) on the first healthcare data.

The method may further include: determining, by the processor, transforms to the residual subspace of the reference profile; receiving, by the processor, second healthcare data including a second plurality of features of at least one second candidate profile; transforming, by the processor using the transforms, the second healthcare data; comparing, by the processor, the transformed second healthcare data to the residual subspace of the reference profile; and detecting, based on the analyzed residual subspace, suspicious behavior of one or more second candidate profiles of the at least one second candidate profile based on a deviation of the at least one second candidate profile within the residual subspace from the expected profile.

According to some embodiments, there is provided a method including: receiving, by a processor, first healthcare data including a first plurality of features of a plurality of first candidate profiles; creating, by the processor, a reference profile of the healthcare data by dimensionally reducing the first plurality of features, the reference profile including a normal subspace and a residual subspace; determining, by the processor, transforms to the residual subspace of the reference profile; receiving, by the processor, second healthcare data including a second plurality of features of at least one second candidate profile; transforming, by the processor using the determined transforms, the second healthcare data; comparing, by the processor, the transformed second healthcare data to the residual subspace of the reference profile; and detecting, based on the comparison, suspicious behavior of the at least one second candidate profiles based on a deviation of the at least one second candidate profile within the residual subspace from the expected profile.

According to some embodiments, there is provided a system including: a processor; and a memory having stored thereon computer program code that, when executed by the processor, controls the processor to: receive first healthcare data including a first plurality of features of a plurality of candidate profiles; identifying a profile calculation window; create a reference profile of the first healthcare data over the profile calculation window by dimensionally reducing the first plurality of features, the reference profile including a normal subspace and a residual subspace; analyze the residual subspace of the reference profile; and detect, based on the analyzed residual subspace, suspicious behavior of one or more candidate profiles of the plurality of candidate profiles based on a deviation of the one or more first candidate profiles within the residual subspace from an expected profile.

The computer program code, when executed by the processor, may further control the processor to: determine a plurality of derived features from the first plurality of features based on a preliminary analysis of the healthcare data.

The computer program code, when executed by the processor, may further control the processor to: detect the suspicious behavior by determining, based on the analyzed residual subspace, a deviation level of the one or more first candidate profiles, and in response to a deviation level of a candidate profile of the one or more first candidate profiles exceeding a threshold, automatically flag the suspicious behavior of the candidate profile of the one or more first candidate profiles.

The computer program code, when executed by the processor, may further control the processor to: detect the suspicious behavior by determining, based on the analyzed residual subspace, a deviation level of the one or more first candidate profiles, and in response to a deviation level of a candidate profile of the one or more first candidate profiles exceeding a threshold, automatically stop a payment for a claim related to the candidate profile of the one or more first candidate profiles.

The computer program code, when executed by the processor, may further control the processor to: detect the suspicious behavior by determining, based on the analyzed residual subspace, a deviation level of the one or more first candidate profiles; determine respective costs associated with the one or more first candidate profiles; combine the respective deviation levels and the respective costs to determine respective expected values of the detected suspicious behavior; and rank the one or more first candidate profiles based on the respective expected values. The computer program code, when executed by the processor, may further control the processor to: calculate deviation values of the detected one or more first candidate profiles; and rank the detected one or more first candidate profiles based on the calculated deviation values.

The computer program code, when executed by the processor, may further control the processor to determine a basis for the ranking by calculating, by the processor, a normalized joint probability between a combination of categorical values within the first healthcare data to identify uncommon combinations.

The computer program code, when executed by the processor, may further control the processor to: determine a basis for the ranking by: identifying, by the processor, unusual numerical values within the first healthcare data; identifying, by the processor, uncommon categorical values within the first healthcare data; and calculating, by the processor, a normalized joint probability between a combination of categorical values within the first healthcare data to identify uncommon combinations, and provide the ranking and the identified numerical values, the uncommon categorical values, and the identified uncommon combinations for additional healthcare analysis.

The computer program code, when executed by the processor, may further control the processor to dimensionally reduce the plurality of features by performing, by the processor, Principal Component Analysis (PCA) on the healthcare data.

The computer program code, when executed by the processor, may further control the processor to: determine transforms to the residual subspace of the reference profile; receive second healthcare data including a second plurality of features of at least one second candidate profile; transform, using the determined transforms to the residual space, the second healthcare data; compare the transformed second healthcare data to the residual subspace of the reference profile; and detect, based on the comparison, suspicious behavior of one or more second candidate profiles of the at least one second candidate profile based on a deviation of the at least one second candidate profile within the residual subspace from the expected profile.

The computer program code, when executed by the processor, may further control the processor to: output, for display, a user interface configured to receive an indication for adjusting the profile calculation window.

### BRIEF DESCRIPTION OF THE FIGURES

The accompanying drawings illustrate one or more embodiments and/or aspects of the disclosure and, together with the written description, serve to explain the principles of the disclosure. Wherever possible, the same reference numbers are used throughout the drawings to refer to the same or like elements of an embodiment, and wherein:

FIG. 1 is a flow chart of identifying suspicious healthcare behavior according to an example embodiment.

FIG. 2 is a flow chart of dimensional reduction according to an example embodiment.

FIGS. 3A-3E are flow charts of post-analysis according to one or more example embodiments.

FIG. 4 is a flow chart of identifying suspicious healthcare behavior according to an example embodiment.

FIG. 5 is a flow chart of identifying suspicious healthcare behavior according to an example embodiment.

FIG. 6 is an example computer architecture for implementing example embodiments.

### DETAILED DESCRIPTION

According to some implementations of the disclosed technology, suspicious behavior in healthcare may be detected through analysis of a set of healthcare data. The method may be executed without input as to what types of behaviors or profiles would be considered suspicious. In some cases, a ranking or risk score of most-suspicious behavior may be provided. In some embodiments, the method may automatically adapt to changes in treatment patterns or coding systems.

According to some embodiments, the method may include extracting features from healthcare data to create a reference profile. A risk score can be generated based on a level of conformity between candidate profiles and the reference profile. In some cases, based on a risk score, a claim may be automatically accepted or denied. In some cases, based on a risk score, claim payments may be automatically stopped.

Some embodiments may be used with other prospective or retrospective claims processing systems and techniques. Some embodiments may identify suspicious claims, providers, or beneficiaries.

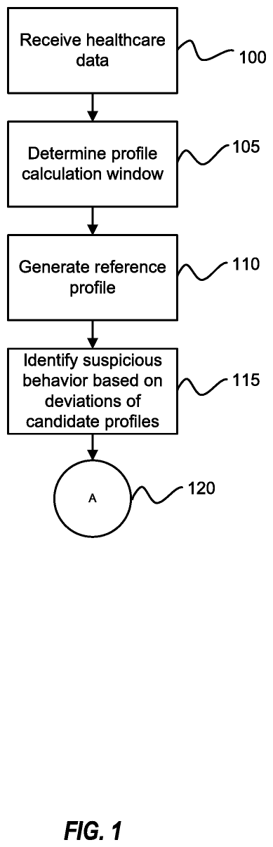

Referring now to the figures, FIG. 1 is a flow chart of identifying suspicious behavior within healthcare according to an example embodiment. As a non-limiting example, the identifying of FIG. 1 may identify suspicious behavior of a healthcare provider from healthcare billing claims, but one of ordinary skill will understand that a similar technique may be provided to identify suspicious behavior at various levels, for example, within individual or groups of claims and/or beneficiaries. As will be understood by one of ordinary skill, a provider may be an individual healthcare provider (e.g., a physician) or may be a larger organization, such as, as non-limiting examples, a hospital, a physician group, or a healthcare center.

Referring to FIG. 1, healthcare data (e.g., healthcare provider data) is received in block 100. In some cases, the healthcare data may be individual or aggregate claim data. The data may correspond to individual beneficiaries, or correspond to providers or services. In some cases, the healthcare data may be partially or fully aggregated.

In block 105, a profile calculation window is determined. The profile calculation window defines a time period over which the healthcare data (e.g., healthcare billing claims) are to be analyzed to create the reference profile. In some situations, the profile calculation window is a rolling window, and the reference profile may be recalculated as the profile window changes (e.g., periodically or on demand) based on claims presented during the rolling window. In some cases, a larger profile window reduces the effect of temporary changes in filing patterns within the reference profile. However, certain types of suspicious behavior may be more easily detected using a shorter profile calculation window. For example, suspicious behavior related to phantom clinics may not be identified if the profile calculation window spans larger than the few months the clinics are typically in operation. In some embodiments, a user interface may be presented to a user for setting or selecting the profile calculation window. In some cases, the reference profile may be considered a dynamic profile, which may change as the profile window changes. In some cases, the reference profile may be self-adapting based on changes to the supplied healthcare data. In some embodiments, healthcare data that falls outside of the calculation window may be excluded from further analysis.

In some embodiments, healthcare filters may be applied. As non-limiting example, the healthcare data may be filtered by provider type (e.g., specialty or organization), provider or service locality (e.g., state or zip code), procedure cost, and service data. One of ordinary skill will understand that these are merely examples, and fewer, additional, or alternative healthcare filters may be applied before generating a reference profile.

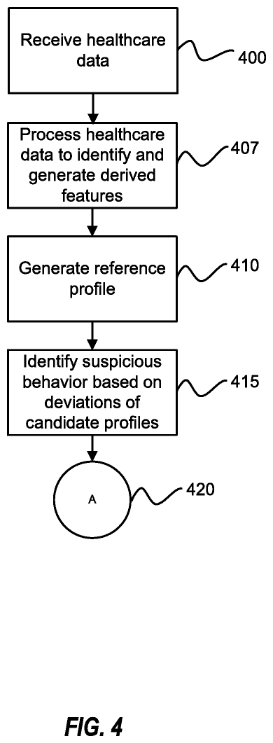

Next, the reference profile is generated in block 110 from features of the healthcare data. The features are measurable properties of healthcare claims. The feature may be used to determine underlying patterns which providers follow while performing their duties. In some cases, the features will depend on the available healthcare data. In some cases, the features may be determined a priori or through a preliminary analysis of the healthcare data, as will be discussed below in greater detail with reference to FIG. 4. In some cases, a reference profile is generated for each type of healthcare provider (e.g., general practitioner, cardiologist, psychiatrist, pediatrician, etc.). To generate the reference profile, a dimensionality reduction technique is applied to the features of the providers over the calculation window. The features may be aggregated (e.g., a total number of claims over the calculation window) or non-aggregated (e.g., elements of individual claims).

For example, a dimensionality reduction technique known as Principal Component Analysis (PCA) may be used to determine principal components (PC) of the dataset of a particular provider type. The number of PCs is generally less than the original number of variables. With PCA, the first PC captures the largest variance along a single axis, and subsequent PCs capture the largest possible variance along the remaining orthogonal directions. This gives an ordered sequence of PCs with decreasing amount of variance along each orthogonal axis.

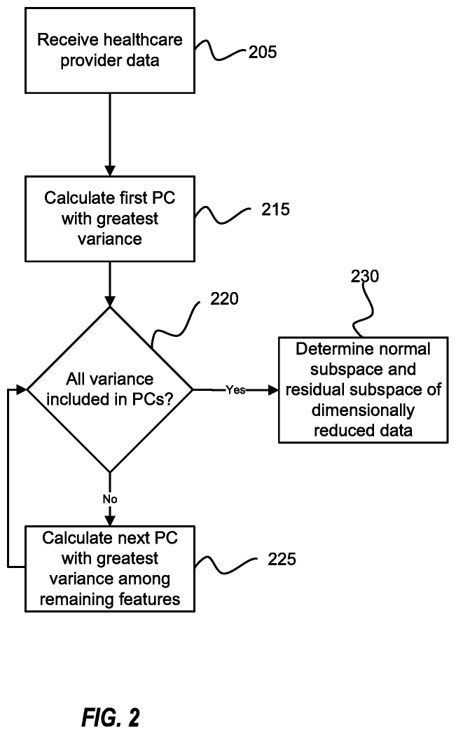

An example process of PCA is illustrated in FIG. 2. As a non-limiting example, the process of PCA described with reference to FIG. 2 may be applied to healthcare provider data, one or ordinary skill will understand that this is merely an example and PCA may be used on other healthcare data, such as claims or beneficiaries.

Referring to FIG. 2, provider data of a particular provider type t is received in block 205. Next, in block 215, a first PC having a largest variance is calculated. Then, if some variance (i.e., data) is not covered by the current set of PCs (220-No), a next PC having a largest variance among the remaining orthogonal dimensions is calculated in block 225. Once all variance is accounted for within the collection of PCs (220-Yes), normal and residual subspaces of the PCs are determined in block 230.

For illustrative purposes, performing PCA on a collection of provider data with features f₁-fᵢ, over time window w yields n PCs as follows:

PCA ([f₁, f₂, . . . , fᵢ]_{w}^{t})→PC₁^{t}(w), PC₂^{t}(w), . . . , PCₙ^{t}(w).

Once the resultant PCs are obtained from the PCA performed on the provider data, the amount of variance captured by each PC is determined Typically, a small subset of the first k PCs capture most of the data. These PCs tend to have a much larger variance than the remaining PCs, and form a normal subspace p^{t}(w):

p^{t}(w)=PC₁^{t}(w), PC₂^{t}(w), . . . , PCₖ^{t}(w).

The normal subspace p^{t}(w) explains the predominant normal behavior of providers over time window w, and may be referred to as the reference profile. The remaining PCs form the residual subspace p̅^̅{̅t̅}̅(̅w̅)̅:

p^{t}(w)=PC₍ₖ₊₁₎^{t}(w), PC₍ₖ₊₂^{t}(w), . . . , PCₙ^{t}(w).

As the normal subspace p^{t}(w) has the greatest variance, the majority of conforming behavior will be defined by the normal subspace p^{t}(w). In some related art techniques, the normal subspace p^{t}(w) would be analyzed to detect providers that deviate from the norm and identify suspicious behavior, and the residual subspace p̅^̅{̅t̅}̅(̅w̅)̅ would be discarded. By discarding the residual subspace p̅^̅{̅t̅}̅(̅w̅)̅, these related art techniques may not identify many instances of suspicious behavior. Although PCA is described with reference to FIG. 2, one of ordinary skill will understand that alternative dimensionality reduction techniques are within the scope of the present disclosure. For example, in some embodiments, single value decomposition (SVD) and other currently known or subsequently developed techniques may be used to create a residual subspace without departing from the scope of the present disclosure.

Referring back to FIG. 1, in block 115, the residual subspace p̅^̅{̅t̅}̅(̅w̅)̅ is analyzed to identify suspicious behavior. In some cases, if a large component of a provider's behavior cannot be described in terms of more common provider behavior (i.e., is not contained in the normal subspace p^{t}(w)), then the provider would have a relatively large component in the residual subspace p̅^̅{̅t̅}̅(̅w̅)̅, which would indicate a deviation from the reference profile and suspicious behavior. In some embodiments, deviations of provider candidate profiles within the residual subspace p̅^̅{̅t̅}̅(̅w̅)̅ are determined and used to identify suspicious behavior.

As a non-limiting example, consider a provider that makes claims for 100 patients. If 90 of the patients are treated normally (e.g., 10 patients are treated suspiciously), most of the provider's activity may conform to the normal subspace. Certain related art techniques may not identify the suspicious behavior related to the 10 patients, which would be contained in the residual subspace. However, a method or system according to some embodiments would identify the suspicious behavior related to the 10 patients through examination of the residual subspace.

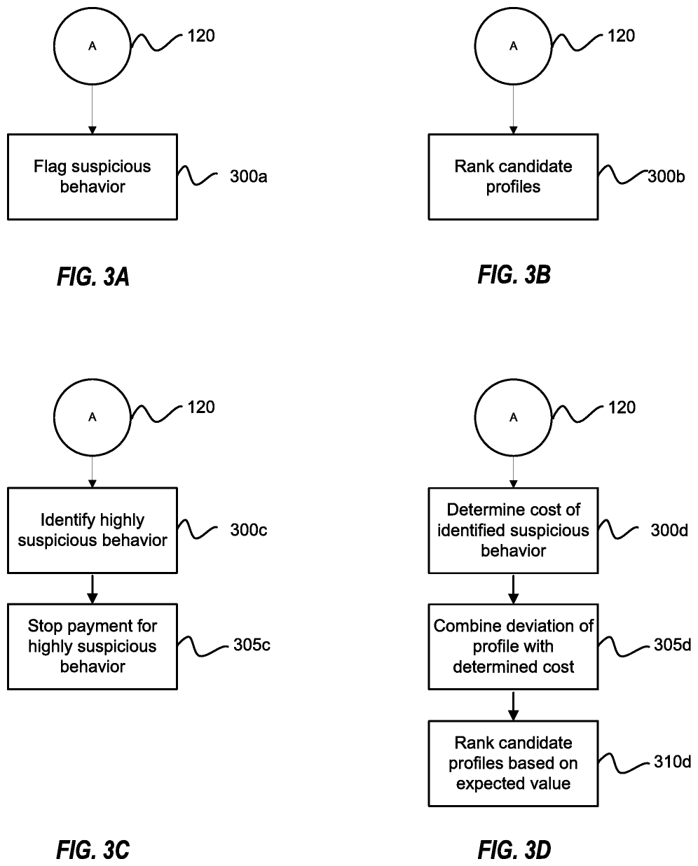

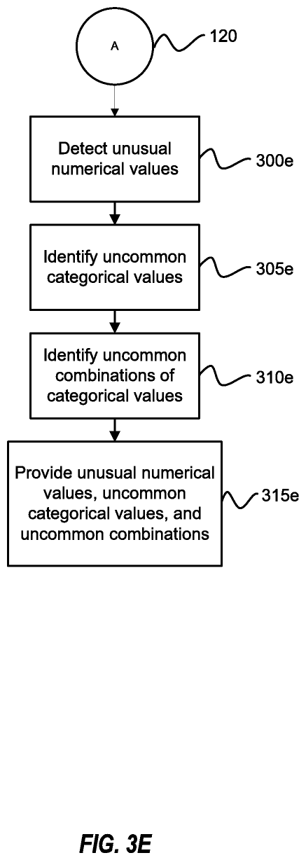

Once suspicious behavior is identified in block 115, post-analysis may begin in block 120. FIGS. 3A-3E are flowcharts of post analysis processes according to some example embodiments.

Referring to FIG. 3A, the post processing 120 may include flagging suspicious behavior (e.g., flagging healthcare provider profiles containing the identified suspicious behavior) in block 300a. For example, information on the identified suspicious behavior may be forwarded to an analyst for additional review. In some cases, risk scores or relative ranking may be provided with the flagged profiles. In some embodiments, particular unusual numerical values or uncommon categorical values within the provider data may be identified as a basis for the identification of suspicious behavior or the risk score, and provided along with the flagged profiles.

Referring to FIG. 3B, the post processing 120 may include ranking the profiles for which suspicious behavior has been identified in block 300b. For example, the profiles with a greater deviation (e.g., those having a greater amount of behavior defined within the residual subspace) may be ranked higher. In some cases, the suspicious candidate profiles may be presented in the ranked order. In some embodiments, only a subset of the suspicious candidate profiles may be presented. For example, only candidate profiles having suspicious behavior within a most recent period of the profile window may be presented. In some embodiments, only a certain number of the most suspicious profiles may be presented. In some cases, as the candidate profile window and the most recent period of time changes, the ranking may change. For example, for a candidate profile window of 10 days, if 1000 claims are received each day from days 1 through 10, the 200 most suspicious claims from days 1 through 10 may be identified on day 11. If 1000 more claims are received on day 11, the 200 most suspicious claims of the 10000 claims received from days 2 through 11 may be identified on day 11. In some cases, previously identified suspicious behavior may be excluded from subsequent rankings.

Referring to FIG. 3C, the post processing 120 may include identifying highly suspicious behavior in block 300c. For example, profiles having a deviation greater than a threshold (e.g., having an amount of behavior defined within the residual subspace) may be identified as highly suspicious. Then, in block 305c, payments related to the highly suspicious behavior may be automatically halted.

Referring to FIG. 3D, the post processing 120 may include determining a cost of the profiles for which suspicious behavior has been identified in block 300d. For example, in the case of the profile being a healthcare claim profile, the cost may be an agreed upon cost of a procedure. As another, non-limiting example, if the profile is a provider profile, the cost may correlate to aggregate expected payments to the provider, or may be based on a subset of payments for individual claims identified as containing suspicious behavior. Then, in block 305d, the determined cost is combined with a suspiciousness of the behavior (e.g., a deviation from an expected profile). Based on the combination (e.g., an expected value of investigating the suspicious behavior), the profiles are ranked in block 310d.

FIG. 3E is a flowchart of identifying a basis for an identification of suspicious behavior of a provider according to an example embodiment. Referring to FIG. 3E, in block 300e, unusual numerical values may be detected using one of a variety of statistical techniques, as would be known by one of ordinary skill. In block 305e, the presence of uncommon categorical (i.e., non-numeric) values are identified within a specific provider fields.

In block 310e, combinations of categorical values are examined to identify the presence of unusual combinations. For example, in some cases, combinations between a provider type and a categorical field value may be used to determine whether a field value is uncommon for the provider type:
c(<provide_type>, <categorical_field_value>).
   In other cases, two categorical field values within the same provider may be analyzed to determine if the presence of both categorical fields is uncommon:
c(<first_categorical_field_value>, <second_categorical_field_value>).
   For example, combinations of two different procedures may be analyzed to determine whether it is unusual for both procedures to occur together.

To calculate the commonality function, the joint probability of both input values is calculated. However, since healthcare data for providers, claims, or beneficiaries may have high arity (i.e., having a large number of features), the combination of any two fields may often be relatively rare, and thus may appear anomalous. Accordingly, in some aspects of the disclosure, the joint probability is normalized by dividing the joint probability by the marginal probability of both attributes:

$$c ( a , b ) = \frac{P ( a , b )}{P ( a ) P ( b )} ,$$

where b is a first categorical value, and a is either a provider type or a second categorical value. A smaller value of c signifies that a and b do not usually co-occur, and their combination is an anomaly.

In block 315e, unusual numerical values, uncommon categorical values, and uncommon combinations are provided. For example, such information may be provided, along with the flagged providers, claims, or beneficiaries, to a fraud analyst for further analysis and investigation.

As non-limiting example implementation of the technique described with reference to FIG. 3E, consider the claims presented in Table 1 below:

TABLE 1
Example Claims Fired by Various Dermatologists

| | | Field 1 | Field 2 | Field 3 | Field 4 |
|---|---|---|---|---|---|
| | | (Cate- | (Numer- | (Numer- | (Cate- |
| Rank | Claim | gorical) | ical) | ical) | gorical) |
| 1 | Claim A | A1 | A2 | A3 | A4 |
| 2 | Claim B | B1 | B2 | B3 | B4 |
| 3 | Claim C | C1 | C2 | C3 | C4 |
| 4 | Claim D | D1 | D2 | D3 | D4 |

As example applications of the commonality functions for Claim A in Table 1, we have:

$${c}_{1} ( dermatologist , A 1 ) = \frac{P ( dermatologist , A 1 )}{P ( dematologist ) P ( A 1 )}$$

$${c}_{2} ( dermatologist , A 4 ) = \frac{P ( dermatologist , A 4 )}{P ( dermatologist ) P ( A 4 )} . \\ {c}_{3} ( A 1 , A 4 ) = \frac{P ( A 1 , A 4 )}{P ( A 1 ) P ( A 4 )}$$

Commonality functions c₁ and c₂ check whether it is unusual for a dermatologist (i.e., provider type) to have a claim that includes A1 and A4, respectively. Commonality function c₃ checks whether it is unusual for a single claim to include both A1 and A4. Although FIG. 3E has been discussed with regards to bases for suspicious behavior identification of a provider, one of ordinary skill will understand that a similar technique may be provided to identify bases for suspicious behavior identification of individual or groups of claims and/or beneficiaries.

One of ordinary skill will understand that elements of the post analysis processes described with reference to FIGS. 3A-3E may be combined in certain embodiments where not mutually exclusive. For example, the rankings produced in 305b and 305d may be provided along with the flagged suspicious profiles.

Referring to FIG. 4, FIG. 4 is a flow chart of a method for identifying suspicious healthcare behavior according to an example embodiment. The method includes receiving healthcare data (block 400), processing the healthcare data to identify and generate derived features (block 407), generating a reference profile based on the derived features (block 410), identifying suspicious behavior based on deviations of candidate profiles (block 415), and performing post analysis (block 420). Blocks 400, 410, 415, and 420 may be similar to the corresponding elements discussed above with reference to FIG. 1. Accordingly, a detailed description of these elements will not be repeated for compactness.

Referring to block 407, in some embodiments, the received healthcare data is processed to identify and generate derived features. The derived features may include certain features inherent in the healthcare data (e.g., procedure codes). In some cases, the derived features may include combinations of features within the healthcare data. For example, some derived features may be generated by combining a plurality of numerical features using a formula. As another example, numerical and/or categorical features may be combined to reflect patterns of care either within a single claim, for a single beneficiary, or by a healthcare provider. In some cases, the processing in block 407 may include excluding certain features of the healthcare data from further analysis (e.g., removing or ignoring these features when generating the reference profile.)

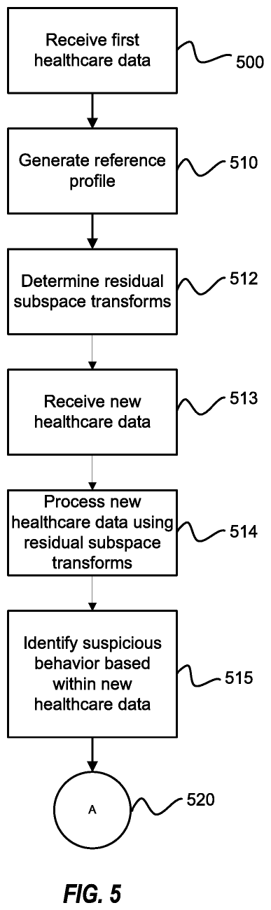

Referring to FIG. 5, FIG. 5 is a flow chart of a method for identifying suspicious healthcare behavior according to an example embodiment. The method includes receiving first healthcare data (block 500), generating a reference profile from the healthcare data (block 510), determining residual subspace transforms (block 512), receiving new healthcare data (block 513), processing the new healthcare data using the residual subspace transforms (block 514), identifying suspicious behavior (block 515), and performing post analysis (block 520). Blocks 500, 510, 515, and 520 may be similar to the corresponding elements discussed above with reference to FIG. 1. Accordingly, a detailed description of these elements will not be repeated for compactness.

Referring to block 512, in some embodiments, transforms for converting features of healthcare data to the residual subspace components may be determined (block 512). These transforms may be mathematical formulas that take the analyzed features of healthcare data and map it to the residual subspace. In some cases, transforms for converting features of healthcare data to the normal subspace may also be determined.

In block 513, new healthcare data is received. The new healthcare data may be of a same type as the first healthcare data used to generate the reference profile. As a non-limiting example, if the first healthcare data is provider healthcare data, the new healthcare data may also be provider healthcare data.

In block 514, the new healthcare data is processed using the residual subspace transforms. In other words, the new healthcare data is transformed into the same dimensions as the reference profile, so that suspicious behavior may be identified in block 515, for example, by comparison within the residual space.

In some embodiments, suspicious behavior may be identified from the within the first healthcare data and within the new healthcare data. In some embodiments, the first healthcare data may be analyzed based on a rolling profile window, and a reference profile may be repeatedly generated from the first healthcare data and new healthcare data within the rolling window. As non-limiting examples, the reference profile may be generated periodically (e.g., daily or weekly), routinely, or in response to a user command In some cases, new healthcare data received between generations of reference profiles may be processed using the residual subspace transforms in order to detect suspicious behavior within the new healthcare data (e.g., new claims).

In some embodiments, techniques disclosed in the present disclosure require no user input. Rather, the techniques may identify suspicious behavior based solely on analysis of the healthcare data.

In some embodiments, healthcare data may be taken directly from forms or other submissions of providers or beneficiaries. In such cases, optical character recognition or other techniques known to one of ordinary skill may be used to extract healthcare data from the submissions.

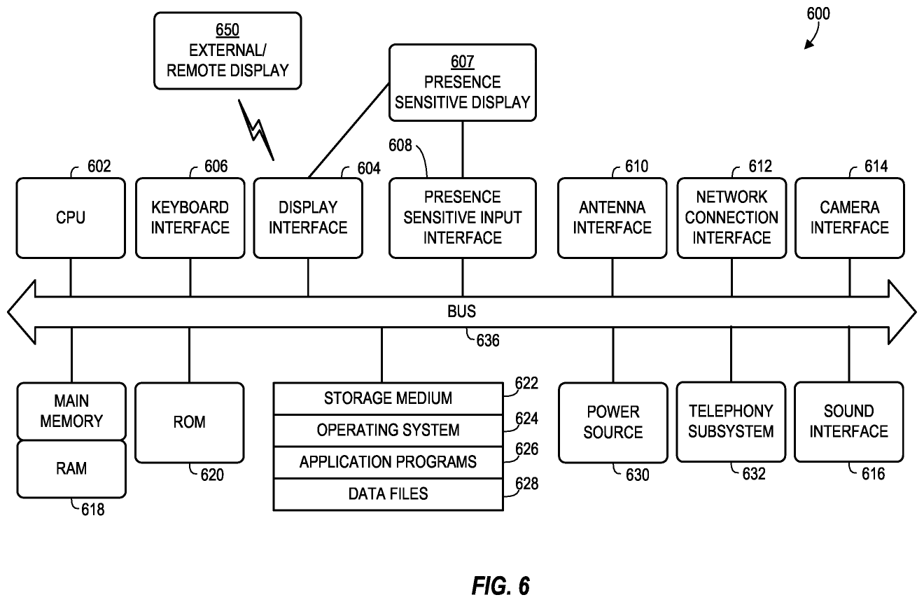

FIG. 6 is a block diagram of an illustrative computer system architecture 600, according to an example implementation. For example, the computer system architecture 600 may implement one or more techniques within the scope of the present disclosure. It will be understood that the computing device architecture 600 is provided for example purposes only and does not limit the scope of the various implementations of the present disclosed systems, methods, and computer-readable mediums.

The computing device architecture 600 of FIG. 6 includes a central processing unit (CPU) 602, where computer instructions are processed, and a display interface 604 that acts as a communication interface and provides functions for rendering video, graphics, images, and texts on the display. In certain example implementations of the disclosed technology, the display interface 604 may be directly connected to a local display, such as a touch-screen display associated with a mobile computing device. In another example implementation, the display interface 604 may be configured for providing data, images, and other information for an external/remote display 650 that is not necessarily physically connected to the mobile computing device. For example, a desktop monitor may be used for minoring graphics and other information that is presented on a mobile computing device. In certain example implementations, the display interface 604 may wirelessly communicate, for example, via a Wi-Fi channel or other available network connection interface 612 to the external/remote display 650.

In an example implementation, the network connection interface 612 may be configured as a communication interface and may provide functions for rendering video, graphics, images, text, other information, or any combination thereof on the display. In one example, a communication interface may include a serial port, a parallel port, a general purpose input and output (GPIO) port, a game port, a universal serial bus (USB), a micro-USB port, a high definition multimedia (HDMI) port, a video port, an audio port, a Bluetooth port, a near-field communication (NFC) port, another like communication interface, or any combination thereof. In one example, the display interface 604 may be operatively coupled to a local display, such as a touch-screen display associated with a mobile device. In another example, the display interface 604 may be configured to provide video, graphics, images, text, other information, or any combination thereof for an external/remote display 650 that is not necessarily connected to the mobile computing device. In one example, a desktop monitor may be used for mirroring or extending graphical information that may be presented on a mobile device. In another example, the display interface 604 may wirelessly communicate, for example, via the network connection interface 612 such as a Wi-Fi transceiver to the external/remote display 650.

The computing device architecture 600 may include a keyboard interface 606 that provides a communication interface to a keyboard. In one example implementation, the computing device architecture 600 may include a presence-sensitive display interface 608 for connecting to a presence-sensitive display 607. According to certain example implementations of the disclosed technology, the presence-sensitive display interface 608 may provide a communication interface to various devices such as a pointing device, a touch screen, a depth camera, etc. which may or may not be associated with a display.

The computing device architecture 600 may be configured to use an input device via one or more of input/output interfaces (for example, the keyboard interface 606, the display interface 604, the presence sensitive display interface 608, network connection interface 612, camera interface 614, sound interface 616, etc.) to allow a user to capture information into the computing device architecture 600. The input device may include a mouse, a trackball, a directional pad, a track pad, a touch-verified track pad, a presence-sensitive track pad, a presence-sensitive display, a scroll wheel, a digital camera, a digital video camera, a web camera, a microphone, a sensor, a smartcard, and the like. Additionally, the input device may be integrated with the computing device architecture 600 or may be a separate device. For example, the input device may be an accelerometer, a magnetometer, a digital camera, a microphone, and an optical sensor.

Example implementations of the computing device architecture 600 may include an antenna interface 610 that provides a communication interface to an antenna; a network connection interface 612 that provides a communication interface to a network. As mentioned above, the display interface 604 may be in communication with the network connection interface 612, for example, to provide information for display on a remote display that is not directly connected or attached to the system. In certain implementations, a camera interface 614 is provided that acts as a communication interface and provides functions for capturing digital images from a camera. In certain implementations, a sound interface 616 is provided as a communication interface for converting sound into electrical signals using a microphone and for converting electrical signals into sound using a speaker. According to example implementations, a random access memory (RAM) 618 is provided, where computer instructions and data may be stored in a volatile memory device for processing by the CPU 602.

According to an example implementation, the computing device architecture 600 includes a read-only memory (ROM) 620 where invariant low-level system code or data for basic system functions such as basic input and output (I/O), startup, or reception of keystrokes from a keyboard are stored in a non-volatile memory device. According to an example implementation, the computing device architecture 600 includes a storage medium 622 or other suitable type of memory (e.g. such as RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives), where the files include an operating system 624, application programs 626 (including, for example, a web browser application, a widget or gadget engine, and or other applications, as necessary) and data files 628 are stored. According to an example implementation, the computing device architecture 600 includes a power source 630 that provides an appropriate alternating current (AC) or direct current (DC) to power components.

According to an example implementation, the computing device architecture 600 includes a telephony subsystem 632 that allows the device 600 to transmit and receive sound over a telephone network. The constituent devices and the CPU 602 communicate with each other over a bus 634.

According to an example implementation, the CPU 602 has appropriate structure to be a computer processor. In one arrangement, the CPU 602 may include more than one processing unit. The RAM 618 interfaces with the computer bus 634 to provide quick RAM storage to the CPU 602 during the execution of software programs such as the operating system application programs, and device drivers. More specifically, the CPU 602 loads computer-executable process steps from the storage medium 622 or other media into a field of the RAM 618 in order to execute software programs. Data may be stored in the RAM 618, where the data may be accessed by the computer CPU 602 during execution.

The storage medium 622 itself may include a number of physical drive units, such as a redundant array of independent disks (RAID), a floppy disk drive, a flash memory, a USB flash drive, an external hard disk drive, thumb drive, pen drive, key drive, a High-Density Digital Versatile Disc (HD-DVD) optical disc drive, an internal hard disk drive, a Blu-Ray optical disc drive, or a Holographic Digital Data Storage (HDDS) optical disc drive, an external mini-dual in-line memory module (DIMM) synchronous dynamic random access memory (SDRAM), or an external micro-DIMM SDRAM. Such computer readable storage media allow a computing device to access computer-executable process steps, application programs and the like, stored on removable and non-removable memory media, to off-load data from the device or to upload data onto the device. A computer program product, such as one utilizing a communication system may be tangibly embodied in storage medium 622, which may include a machine-readable storage medium.

According to one example implementation, the term computing device, as used herein, may be a CPU, or conceptualized as a CPU (for example, the CPU 602 of FIG. 6). In this example implementation, the computing device (CPU) may be coupled, connected, and/or in communication with one or more peripheral devices, such as display. In another example implementation, the term computing device, as used herein, may refer to a mobile computing device such as a Smartphone, tablet computer, or smart watch. In this example implementation, the computing device may output content to its local display and/or speaker(s). In another example implementation, the computing device may output content to an external display device (e.g., over Wi-Fi) such as a TV or an external computing system.

In example implementations of the disclosed technology, a computing device may include any number of hardware and/or software applications that are executed to facilitate any of the operations. In example implementations, one or more I/O interfaces may facilitate communication between the computing device and one or more input/output devices. For example, a universal serial bus port, a serial port, a disk drive, a CD-ROM drive, and/or one or more user interface devices, such as a display, keyboard, keypad, mouse, control panel, touch screen display, microphone, etc., may facilitate user interaction with the computing device. The one or more I/O interfaces may be used to receive or collect data and/or user instructions from a wide variety of input devices. Received data may be processed by one or more computer processors as desired in various implementations of the disclosed technology and/or stored in one or more memory devices.

One or more network interfaces may facilitate connection of the computing device inputs and outputs to one or more suitable networks and/or connections; for example, the connections that facilitate communication with any number of sensors associated with the system. The one or more network interfaces may further facilitate connection to one or more suitable networks; for example, a local area network, a wide area network, the Internet, a cellular network, a radio frequency network, a Bluetooth enabled network, a Wi-Fi enabled network, a satellite-based network any wired network, any wireless network, etc., for communication with external devices and/or systems.

According to some implementations, the computer program code may control the computing device to receive healthcare data, identify features of the data and a profile calculation window, generate a reference profile through dimensional reduction, and detect suspicious behavior within the healthcare data based on the reference profile. In some cases, the computer program code may further control the computer device to determine deviations of candidate profiles from the reference profile, rank candidate profiles, and flag suspicious behavior. In some cases, the computer program code may further control the computer device to perform dimensional reduction through a PCA technique. In some cases, the computer program code may further control the computer device to detect unusual numerical values, identify uncommon categorical values, or identify uncommon combinations of categorical values within the candidate profiles.

While certain implementations of the disclosed technology have been described throughout the present description and the figures in connection with what is presently considered to be the most practical and various implementations, it is to be understood that the disclosed technology is not to be limited to the disclosed implementations, but on the contrary, is intended to cover various modifications and equivalent arrangements included within the scope of the appended claims and their equivalents. Although specific terms are employed herein, they are used in a generic and descriptive sense only and not for purposes of limitation.

In the foregoing description, numerous specific details are set forth. It is to be understood, however, that implementations of the disclosed technology may be practiced without these specific details. In other instances, well-known methods, structures and techniques have not been shown in detail in order not to obscure an understanding of this description. References to “one implementation,” “an implementation,” “example implementation,” “various implementation,” etc., indicate that the implementation(s) of the disclosed technology so described may include a particular feature, structure, or characteristic, but not every implementation necessarily includes the particular feature, structure, or characteristic. Further, repeated use of the phrase “in one implementation” does not necessarily refer to the same implementation, although it may.

Throughout the specification and the claims, the following terms should be construed to take at least the meanings explicitly associated herein, unless the context clearly dictates otherwise. The term “connected” means that one function, feature, structure, or characteristic is directly joined to or in communication with another function, feature, structure, or characteristic. The term “coupled” means that one function, feature, structure, or characteristic is directly or indirectly joined to or in communication with another function, feature, structure, or characteristic. The term “or” is intended to mean an inclusive “or.” Further, the terms “a,” “an,” and “the” are intended to mean one or more unless specified otherwise or clear from the context to be directed to a singular form.

As used herein, unless otherwise specified the use of the ordinal adjectives “first,” “second,” “third,” etc., to describe a common object, merely indicate that different instances of like objects are being referred to, and are not intended to imply that the objects so described must be in a -given sequence, either temporally, spatially, in ranking, or in any other manner.

This written description uses examples to disclose certain implementations of the disclosed technology, including the best mode, and also to enable any person of ordinary skill to practice certain implementations of the disclosed technology, including making and using any devices or systems and performing any incorporated methods. The patentable scope of certain implementations of the disclosed technology is defined in the claims and their equivalents, and may include other examples that occur to those of ordinary skill. Such other examples are intended to be within the scope of the claims if they have structural elements that do not differ from the literal language of the claims, or if they include equivalent structural elements with insubstantial differences from the literal language of the claims.

1. A method comprising:
receiving, by a first processor, first healthcare data comprising first features of of at least one first candidate profile;
identifying, by a second processor, a profile calculation window;
creating, by a third processor, a reference profile of the first healthcare data over the profile calculation window by dimensionally reducing the first features, the reference profile comprising a normal subspace and a residual subspace;
analyzing, by a fourth processor, the residual subspace of the reference profile; and
detecting, based on the analyzed residual subspace, suspicious behavior of at least one first candidate profiles based on a deviation of one or more first candidate profiles within the residual subspace from an expected profile.

2. The method of claim 1, wherein the first healthcare data comprises at least one of healthcare provider data, healthcare beneficiary data, and healthcare claim data.

3. The method of claim 1, wherein the first, second, third and fourth processors are the same processor;
wherein detecting suspicious behavior comprises determining, by the same processor and based on the analyzed residual subspace, a deviation level of one or more first candidate profiles; and
wherein the method further comprises, in response to the deviation level of at least one first candidate profile exceeding a threshold, automatically flagging, by the same processor, the suspicious behavior of each such first candidate profile.

4. The method of claim 1, wherein the first, second, third and fourth processors are the same processor;
wherein detecting the suspicious behavior comprises determining, by the same processor and based on the analyzed residual subspace, respective deviation levels of one or more first candidate profiles; and
wherein the method further comprises:
determining, by the same processor, respective costs associated with one or more first candidate profiles;
combining, by the same processor, the respective deviation levels and the respective costs to determine respective expected values of the detected suspicious behavior; and
ranking one or more first candidate profiles based on the respective expected values.

5. The method of claim 1, wherein the first, second, third and fourth processors are the same processor; and
wherein the method further comprising comprises:
calculating, by the same processor, deviation values of the detected one or more first candidate profiles; and
ranking the detected first candidate profiles based on the calculated deviation values.

6. The method of claim 5, further comprising determining a basis for the ranking by calculating, by the same processor, a normalized joint probability between a combination of categorical values within the first healthcare data to identify uncommon combinations.

7. The method of claim 5 further comprising:
determining a basis for the ranking by:
identifying, by the same processor, unusual numerical values within the first healthcare data;
identifying, by the same processor, uncommon categorical values within the first healthcare data; and
calculating, by the same processor, a normalized joint probability between a combination of categorical values within the first healthcare data to identify uncommon combinations; and
providing the ranking and the identified numerical values, the uncommon categorical values, and the identified uncommon combinations for additional healthcare analysis.

8. The method of claim 1, wherein dimensionally reducing comprises performing Principal Component Analysis (PCA) on the first healthcare data.

9. The method of claim 1 wherein the first, second, third and fourth processors are the same processor; and
wherein the method further comprises:
determining, by the same processor, transforms to the residual subspace of the reference profile;
receiving, by the same processor, second healthcare data comprising second features of at least one second candidate profile;
transforming, by the same processor using the transforms, the second healthcare data;
comparing, by the same processor, the transformed second healthcare data to the residual subspace of the reference profile; and
detecting, based on the analyzed residual subspace, suspicious behavior of one or more second candidate profiles based on a deviation of at least one second candidate profile within the residual subspace from the expected profile.

10. A method comprising:
receiving, by a processor, first healthcare data comprising features of at least one first candidate profile;
creating, by the processor, a reference profile of the healthcare data by dimensionally reducing the first features, the reference profile comprising a normal subspace and a residual subspace;
determining, by the processor, transforms to the residual subspace of the reference profile;
receiving, by the processor, second healthcare data comprising second features of at least one second candidate profile;
transforming, by the processor using the determined transforms, the second healthcare data;
comparing, by the processor, the transformed second healthcare data to the residual subspace of the reference profile; and
detecting, based on the comparison, suspicious behavior of at least one second candidate profiles based on a deviation of at least one second candidate profile within the residual subspace from the expected profile.

11. A system comprising:
a processor; and
a memory having stored thereon computer program code that, when executed by the processor, controls the processor to:
receive first healthcare data comprising a first plurality of features of a plurality of candidate profiles;
identifying a profile calculation window;
create a reference profile of the first healthcare data over the profile calculation window by dimensionally reducing the first plurality of features, the reference profile comprising a normal subspace and a residual subspace;
analyze the residual subspace of the reference profile; and
detect, based on the analyzed residual subspace, suspicious behavior of one or more candidate profiles of the plurality of candidate profiles based on a deviation of the one or more first candidate profiles within the residual subspace from an expected profile.

12. The system of claim 11, wherein the computer program code, when executed by the processor, further controls the processor to:
determine a plurality of derived features from the first plurality of features based on a preliminary analysis of the healthcare data.

13. The system of claim 11, wherein the computer program code, when executed by the processor, controls the processor to:
detect the suspicious behavior by determining, based on the analyzed residual subspace, a deviation level of the one or more first candidate profiles, and
in response to a deviation level of a candidate profile of the one or more first candidate profiles exceeding a threshold, automatically flag the suspicious behavior of the candidate profile of the one or more first candidate profiles.

14. The system of claim 11, wherein the computer program code, when executed by the processor, controls the processor to:
detect the suspicious behavior by determining, based on the analyzed residual subspace, a deviation level of the one or more first candidate profiles;
determine respective costs associated with the one or more first candidate profiles;
combine the respective deviation levels and the respective costs to determine respective expected values of the detected suspicious behavior; and
rank the one or more first candidate profiles based on the respective expected values.

15. The system of claim 11, wherein the computer program code, when executed by the processor, further controls the processor to:
calculate deviation values of the detected one or more first candidate profiles; and
rank the detected one or more first candidate profiles based on the calculated deviation values.

16. The system of claim 14, wherein the computer program code, when executed by the processor, further controls the processor to determine a basis for the ranking by calculating, by the processor, a normalized joint probability between a combination of categorical values within the first healthcare data to identify uncommon combinations.

17. The system of claim 15, wherein the computer program code, when executed by the processor, further controls the processor to:
determine a basis for the ranking by:
identifying, by the processor, unusual numerical values within the first healthcare data;
identifying, by the processor, uncommon categorical values within the first healthcare data; and
calculating, by the processor, a normalized joint probability between a combination of categorical values within the first healthcare data to identify uncommon combinations, and
provide the ranking and the identified numerical values, the uncommon categorical values, and the identified uncommon combinations for additional healthcare analysis.

18. The system of claim 11, wherein the computer program code, when executed by the processor, further controls the processor to dimensionally reduce the plurality of features by performing, by the processor, Principal Component Analysis (PCA) on the healthcare data.

19. The system of claim 11, wherein the computer program code, when executed by the processor, further controls the processor to:
determine transforms to the residual subspace of the reference profile;
receive second healthcare data comprising a second plurality of features of at least one second candidate profile;
transform, using the determined transforms to the residual space, the second healthcare data;
compare the transformed second healthcare data to the residual subspace of the reference profile; and
detect, based on the comparison, suspicious behavior of one or more second candidate profiles of the at least one second candidate profile based on a deviation of the at least one second candidate profile within the residual subspace from the expected profile.

20. The system of claim 11, wherein the computer program code, when executed by the processor, further controls the processor to:
output, for display, a user interface configured to receive an indication for adjusting the profile calculation window.
